(19) United States
(12) Patent Application Publication
(10) Pub. No.: US 2024/0261870 A1
(43) Pub. Date: Aug. 8, 2024

(54) MODULAR DRILL WITH CLAMPING FINGERS

(71) Applicant: Kennametal Inc., Latrobe, PA (US)

(72) Inventors: Alan BOOKHEIMER, Greensburg, PA (US); Austin BAER, Irwin, PA (US)

(21) Appl. No.: 18/104,911

(22) Filed: Feb. 2, 2023

(51) Int. Cl. B23B 27/16; B23B 51/00

(52) U.S. Cl. CPC B23B 27/1666; B23B 51/0003

(57) ABSTRACT

A modular drill includes an insert and a main body. The insert has a working end and a retention end. The retention end includes a retention bit. The main body has an attachment end for attachment to a toolholder and a receiving end defining an insert pocket. There is at least one clamping finger disposed in the pocket. The insert is disposed in the insert pocket with the retention bit held in tension with the clamping fingers.

### BACKGROUND

This relates in general to cutting tools, such as drills.

One type of cutting tool is a drill with a removeable/replaceable insert. Such drills typically have a main body or shank portion having one end for attachment to a toolholder, i.e. a mount, and another end for attachment to the removeable tip. Currently such drills include a fastener to retain the insert to the shank. In one example, the shank includes a threaded passageway with a screw disposed therein that cooperates with a screw seat in the insert to retain the insert to the shank when the screw is tightened. In order to change inserts this requires a user to remove the drill from an associated tool holder or mount. It is desired that the user be able to exchange the insert without having to remove the shank from an associated mount while still adequately retaining the insert to the shank during normal drilling applications.

### SUMMARY

This relates more particularly to a modular drill including an insert having a working end and a retention end including a retention bit, and a main body having an attachment end for attachment to a toolholder and a receiving end defining an insert pocket. The main body has at least one clamping finger disposed in the pocket. The insert is disposed in the insert pocket with the retention bit held in tension with the one or more clamping fingers.

In at least one embodiment the clamping fingers terminate in radially extending engagement surfaces and the retention bit has a beveled engagement surface such that when the fingers engage the bit the insert is urged toward the pocket.

In at least one embodiment the main body defines a longitudinal axis extending between the attachment end and the receiving end and includes at least two diametrically opposed circumferential outer main portions extending along the longitudinal axis with at least two diametrically opposed flutes disposed between the outer main portions with the fingers generally extending along the longitudinal axis within the main portions.

In at least one embodiment the fingers are unitarily formed with the main portions of the main body.

In at least one embodiment fluid passageways are formed in the main portions of the body and generally extend along the longitudinal axis.

In at least one embodiment one of the clamping fingers includes a threaded aperture formed in the clamping finger or main body and the drill further includes a screw disposed in threaded engagement in the aperture, where turning of the screw in one direction results in contact of the screw with a clamping finger spreading the finger apart to release the retention bit from the clamping finger.

In at least one embodiment an insert rejection arm extends from one of the fingers such that upon spreading of the fingers the arm urges the insert out of the pocket.

In at least one embodiment, the main body includes a radial passageway extending between the exterior of the main body and the insert pocket and generally aligned between the fingers for insertion of a cam wrench.

In at least one embodiment the drill further includes a cam wrench disposed in the radial passageway for urging of the fingers away from each other by rotation of the wrench.

In at least one embodiment, the cam wrench is configured to urge the insert out of the insert pocket by rotation of the wrench.

In at least one alternative embodiment a modular drill has a main body with an attachment end for attachment to a toolholder and a receiving end defining an insert pocket and includes at least one clamping finger disposed in the pocket for holding an insert having a working end and a retention end including a retention bit in the insert pocket by retaining the retention bit held in tension with the one or more clamping fingers. At least one of the clamping fingers includes or the main body includes a threaded aperture formed in the clamping finger or the main body, and a screw is disposed in the aperture and in threaded engagement with the aperture. Turning of the screw in one direction results in contact of the screw with a clamping finger spreading the finger to release a retention bit of an insert retained in the insert pocket from the clamping finger.

In at least one embodiment the clamping fingers terminate in radially extending engagement surfaces and at least one of the fingers includes an insert rejection arm extending from the one of the fingers.

In at least one embodiment the main body defines a longitudinal axis extending between the attachment end and the receiving end and includes at least two diametrically opposed circumferential outer main portions extending along the longitudinal axis with at least two diametrically opposed flutes disposed between the outer main portions. The fingers generally extend along the longitudinal axis within the main portions, and the main body includes a radial passageway formed in one of the main portions and extends between the exterior of the main body and the insert pocket and is generally aligned with the threaded aperture.

In at least one embodiment, the fingers are unitarily formed with the main portions of the main body.

In at least one embodiment, fluid passageways are formed in the main portions of the body and generally extend along the longitudinal axis.

In at least one other alternative embodiment a modular drill has a main body with an attachment end for attachment to a toolholder and a receiving end defining an insert pocket and has at least one clamping finger disposed in the pocket for holding an insert having a working end and a retention end including a retention bit in the insert pocket by retaining the retention bit held in tension with the one or more clamping fingers. The main body includes a radial passageway extending between the exterior of the main body and the insert pocket and generally aligned between the fingers for receiving a cam wrench for urging the fingers away from each other.

In at least one embodiment the drill further includes a cam wrench disposed in the radial passageway where the fingers are urged away from each other by rotation of the wrench.

In at least one embodiment the cam wrench includes at least one off set end portion for engagement of at least one of the fingers during rotation.

In at least one embodiment the cam wrench includes at least one off set end portion for engagement of a retention bit of an insert disposed in the insert pocket during rotation.

In at least one embodiment the main body defines a longitudinal axis extending between the attachment end and the receiving end and includes at least two diametrically opposed circumferential outer main portions extending along the longitudinal axis with at least two diametrically opposed flutes disposed between the outer main portions. The fingers generally extend along the longitudinal axis within the main portions, and the radial passageway is formed in one of the flutes.

Various aspects will become apparent to those skilled in the art from the following detailed description and the accompanying drawings.

### BRIEF DESCRIPTION OF THE DRAWINGS

FIG. 1 is a front end perspective view of a modular drill according to one embodiment.

FIG. 2 is a top view of the drill of FIG. 1.

FIG. 3 is a side view of the drill of FIG. 1.

FIG. 4 is a front end view of the drill of FIG. 1.

FIG. 5 is a top cross-sectional view of the drill of FIG. 1 taken along line 5-5 in FIG. 4.

FIG. 6 is a side cross-sectional view of the drill of FIG. 1 taken along line 6-6 in

FIG. 4.

FIG. 7 is a front end perspective view of the main body of the drill of FIG. 1.

FIG. 8 is a top view of the main body of FIG. 7.

FIG. 9 is a side view of the main body of FIG. 7.

FIG. 10 is a front end view of the main body of FIG. 7.

FIG. 11 is a top cross-sectional view of the main body of FIG. 7 taken along line 11-11 in FIG. 10.

FIG. 12 is a side cross-sectional view of the main body of FIG. 7 taken along line 12-12 in FIG. 10.

FIG. 13 is a is front end cross-sectional view of the main body of FIG. 7 taken along line 13-13 in FIG. 12.

FIG. 14 is a front end perspective view of the insert of the drill of FIG. 1.

FIG. 15 is a rear end perspective view of the insert of FIG. 14.

FIG. 16 is a front view of the insert of FIG. 14.

FIG. 17 is a side cross-sectional view of the insert of FIG. 14 taken along line 17-17 in FIG. 16.

FIG. 18 is a front view of a main body for a modular drill according to another embodiment.

FIG. 19 is a side cross-sectional view of the main body of FIG. 18 taken along line 19-19 in FIG. 18.

FIG. 20 is a top cross-sectional view of the main body of FIG. 18 taken along line 20-20 in FIG. 18 with insert.

FIG. 21 is a front end cross-sectional view of the main body of FIG. 18 taken along line 21-21 in FIG. 20.

FIG. 22 is a front view of the modular drill of FIG. 20 in a clamped state with a cam tool.

FIG. 23 is a top cross-sectional view of the drill with cam tool of FIG. 22 taken along line 23-23 in FIG. 22.

FIG. 24 is a side cross-sectional view of the drill with cam tool of FIG. 22 taken along line 24-24 in FIG. 23.

FIG. 25 is a side cross-sectional view of the drill with cam tool of FIG. 22 taken along line 25-25 in FIG. 23.

FIG. 26 is a front view of the drill with cam tool of FIG. 22 in an unclamped state.

FIG. 27 is a top cross-sectional view of the drill with cam tool of FIG. 26 taken along line 27-27 in FIG. 26.

FIG. 28 is a side cross-sectional view of the drill with cam tool of FIG. 26 taken along line 28-28 in FIG. 27.

FIG. 29 is a side cross-sectional view of the drill with cam tool of FIG. 26 taken along line 29-29 in FIG. 27.

### DETAILED DESCRIPTION

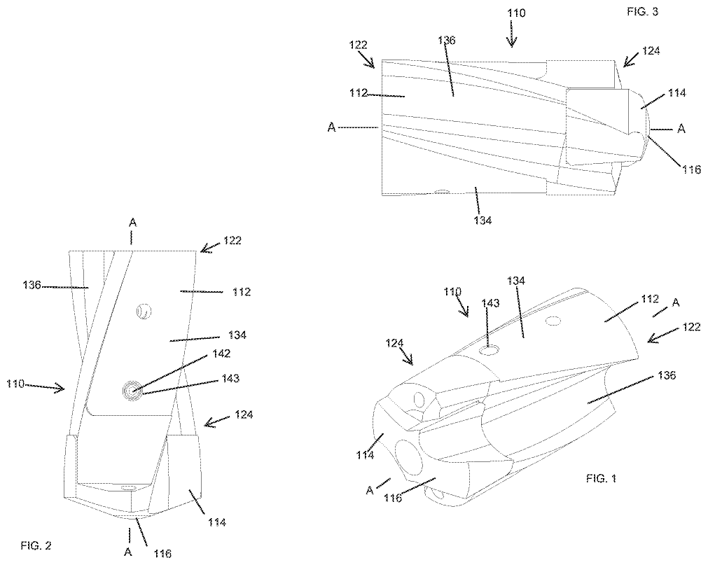

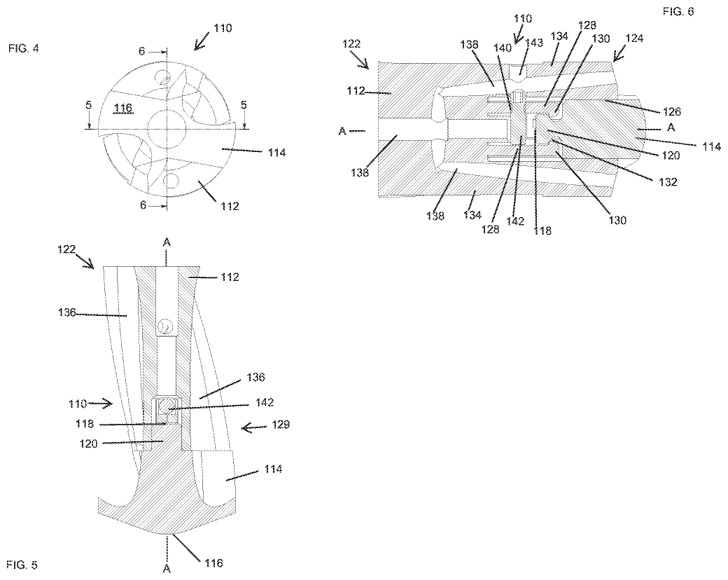

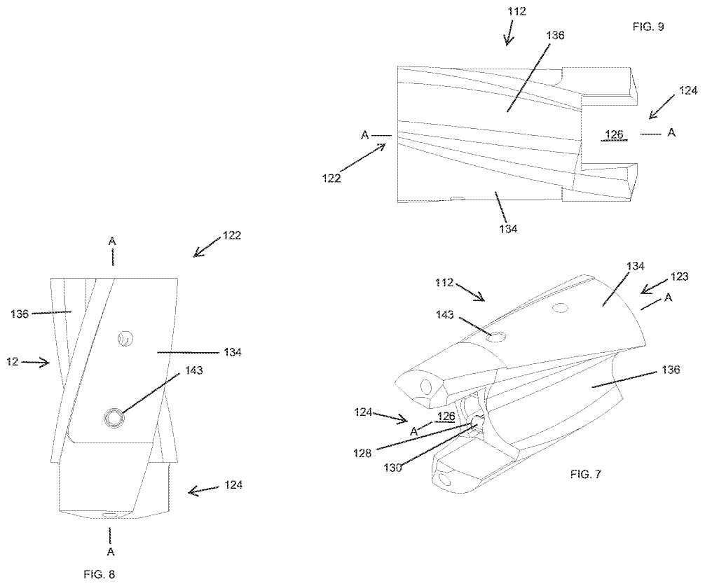

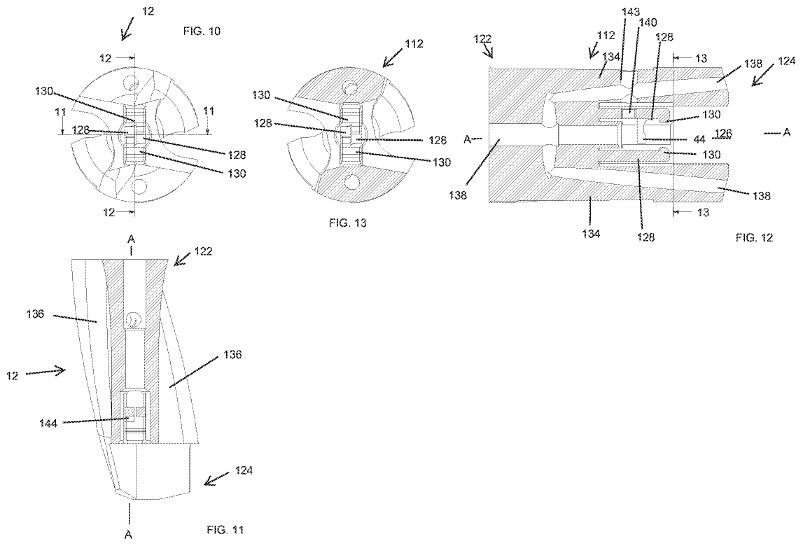

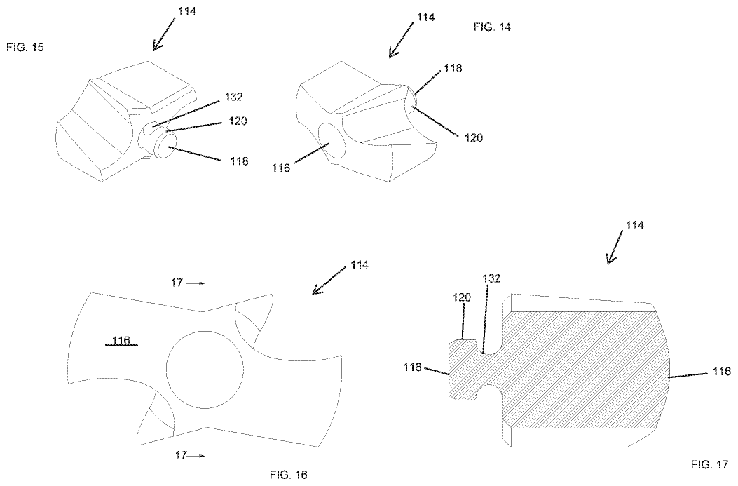

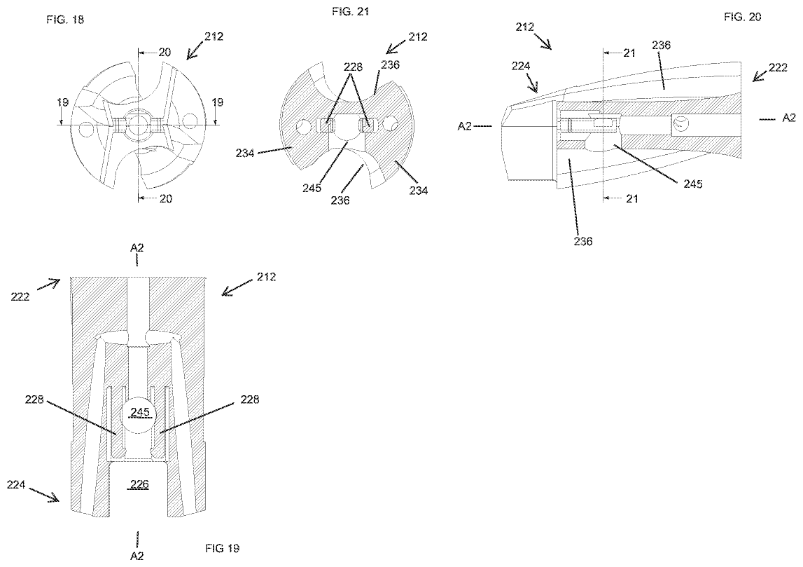

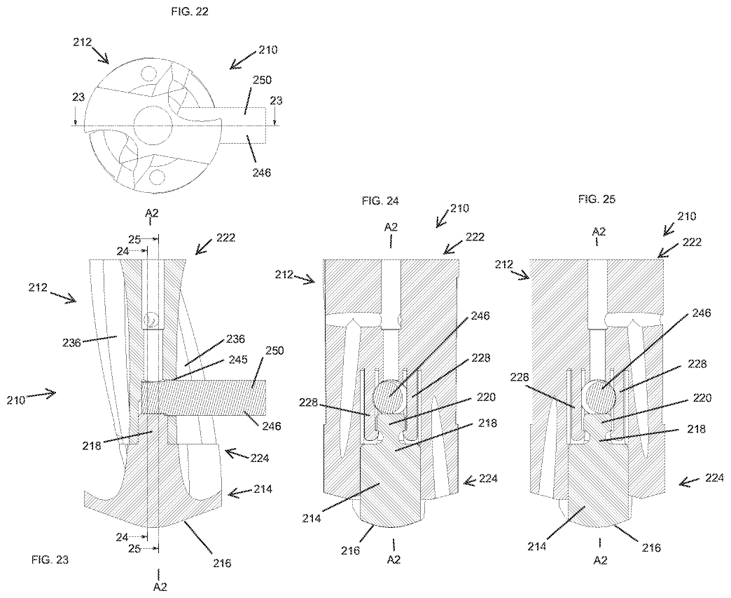

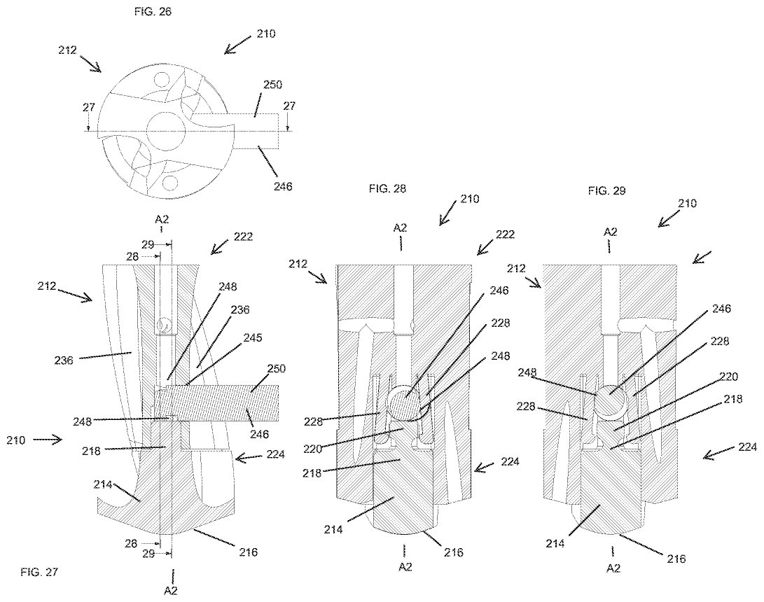

Referring now to the drawings, there is illustrated in FIGS. 1-6 a modular drill 110 including a main body 112, also shown in FIGS. 7-13, and an insert 114, also shown in FIGS. 14-17. With particular reference to FIGS. 14-17, the insert 114 has a working end 116 and a retention end 118 including a retention bit 120. With particular reference to FIGS. 1-13, the main body has an attachment end 122 for attachment to a toolholder (not shown) and a receiving end 124 defining an insert pocket 126. The main body 112 has at least one clamping finger 128, shown with two, disposed in the pocket 126. The insert 114 is disposed in the insert pocket 126 with the retention bit 120 held in tension with the one or more the clamping fingers 128. Optionally, the retention bit 120 may be held between one clamping finger 128 and another portion or the main body 112.

In the illustrated example, the clamping fingers 128 terminate in radially extending engagement surfaces 130 and the retention bit 120 has a beveled engagement surface 132 such that when the fingers 128 engage the retention bit 120 the insert 114 is urged toward the pocket 126. Alternatively or optionally the engagement surfaces 130 have a beveled surface.

In the illustrated example, the main body 112 defines a longitudinal axis A extending between the attachment end 122 and the receiving end 124 and includes at least two diametrically opposed circumferential outer main portions 134 extending along the longitudinal axis A with at least two diametrically opposed flutes 136 disposed between the outer main portions 134 with the fingers 128 generally extending along the longitudinal axis A within the main portions 134.

In at least one exemplary embodiment, the fingers 128 are unitarily formed with the main portions 134 of the main body 112. For further example, the fingers 128 and the main portions 134 may be formed from a common piece of material with the fingers 128 being machined out of the main portions 134. Alternatively, it is contemplated that the fingers 128 may be formed separately and then otherwise joined, secured, or fixed to the main portions 134.

In the illustrated example, the main body 112 further includes optional fluid passageways 138 formed in the main portions 134 of the main body 112 and generally extend along the longitudinal axis A. Such passageways 138 may be utilized to provide coolant, lubricant, and/or wash to the interface between the drill 110 and a workpiece.

In the exemplary embodiment illustrated in FIGS. 1-13, one of the clamping fingers 128 includes a threaded aperture 140 formed in the clamping finger 128. Alternatively, the threaded aperture 140 may be formed in the main body 112. The drill 110 further includes a screw 142 disposed in threaded engagement in the aperture 140 such that turning of the screw 142 in one direction results in contact of the screw 142 with a clamping finger 128, thus, spreading the finger 128 to release the retention bit 120 from the clamping finger 128. Turing of the screw 142 in the opposite direction then moves the screw 142 away from the clamping finger 128 allowing the clamping finger 128 to engage the retention bit 120 and retain the insert 114 in the pocket 126.

Further, in this exemplary embodiment, the main body 112 includes a radial passageway 143 formed in one of the main portions 134 that extends between the exterior of the main body 112 and the insert pocket 126 and is generally aligned with the threaded aperture 140 to provide access to the screw 142.

As best shown in FIGS. 11 and 12, there is also illustrated in the exemplary embodiment an optional insert rejection arm 144 that extends from at least one of the fingers 128 such that upon spreading of the fingers 128 the arm 144 urges the insert 114 out of the pocket 126.

There is shown in FIGS. 18-29 an exemplary alternative embodiment of a modular drill 210. Elements of the alternative drill 210 that are similar to the drill 110 described above are labeled with similar numeric identifiers. The drill 210 has a main body 212 with an attachment end 222 for attachment to a toolholder (not shown) and a receiving end 224. The receiving end 224 defines an insert pocket 226. The drill 210 has at least one clamping finger 228, shown with two, disposed in the pocket 226 for holding an insert 214. The insert 214 has a working end 216 and a retention end 218. The retention end 218 including a retention bit 220 in the insert pocket 226 and retaining the retention bit 220 in tension with the one or more clamping fingers 228. Optionally, the retention bit 220 may be held between one clamping finger 228 and another portion of the drill 210.

In the present example, the main body 212 defines a longitudinal axis A2 extending between the attachment end 222 and the receiving end 224. The main body 212 includes at least two diametrically opposed circumferential outer main portions 234 extending along the longitudinal axis A2 with at least two diametrically opposed flutes 236 disposed between the outer main portions 234. The fingers 228 generally extend along the longitudinal axis A2 within the main portions 234.

The main body 212 includes a radial passageway 245 formed in one of the flutes 236. The radial passageway extends between the exterior of the main body 212 and the insert pocket 226 and is generally aligned between the fingers 228 for receiving a cam wrench 246 for urging the fingers 228 away from each other.

In the exemplary illustrated embodiment, the cam wrench 246 includes at least one off set end portion 248 for engagement of at least one of the fingers 228 during rotation to push the clamping finger 228 radially outward. Additionally, or alternatively, one or more off set portions 248 may engage the retention bit 220 of an insert 214 disposed in the insert pocket 226 during rotation to urge the insert 214 out of the pocket 226.

For example, the cam wrench 246 may be formed from a single piece of material where portions have been removed, for example by machining, to produce the off set portions 248. Alternatively, it is contemplated that the cam wrench 246 may be formed with the off set portions 248, for example by casting or additive manufacturing.

For example, in operation the cam wrench 246 may be inserted into the main body 212 while the insert 214 is in a clamped state, see FIGS. 22-25. The cam wrench 246 may then be rotated for one or more off set portions 248 to engage one or more finger 228 and/or the retention bit 220 to release the insert 214 from the pocket 226, see FIGS. 26-29.

In the illustrated example, the cam wrench 246 is shown as a separable piece from the main body 212 and includes a shaft, bit or handle 250 extending away from the main body 212. It is contemplated, however, that the cam wrench 246 may be permanently or semi-permanently mounted within the radial passageway 245 and preferably terminating at or before the surface of the flute 236. In such a case, the terminated end of the cam wrench 246 may have an engagement, such as a Star, Torx, Allen, Hex, or other tool engagement, formed there in for cooperation with a reciprocal tool to turn the cam wrench 246.

While principles and modes of operation have been explained and illustrated with regard to particular embodiments, it must be understood, however, that this may be practiced otherwise than as specifically explained and illustrated without departing from its spirit or scope.

What is claimed is:

1. A modular drill comprising:
an insert having a working end and a retention end including a retention bit; and
a main body having an attachment end for attachment to a toolholder and a receiving end defining an insert pocket and having one or more clamping fingers disposed in the pocket,
where the insert is disposed in the insert pocket with the retention bit held in tension by the one or more clamping fingers.

2. The drill of claim 1 where the clamping fingers terminate in radially extending engagement surfaces and where the retention bit has beveled engagement surface such that when the fingers engage the bit the insert is urged toward the pocket.

3. The drill of claim 1 where the main body defines a longitudinal axis extending between the attachment end and the receiving end and includes at least two diametrically opposed circumferential outer main portions extending along the longitudinal axis with at least two diametrically opposed flutes disposed between the outer main portions where the fingers generally extend along the longitudinal axis within the main portions.

4. The drill of claim 1 where the fingers are unitarily formed with the main portions of the main body.

5. The drill of claim 3 where fluid passageways are formed in the main portions of the body and generally extend along the longitudinal axis.

6. The drill of claim 1 where a threaded aperture is formed in one of the main body or one of the one or more clamping fingers and where the drill further comprises a screw disposed in threaded engagement in the aperture, where turning of the screw in one direction results in contact of the screw with one of the one or more clamping fingers spreading the finger to release the retention bit from the clamping finger.

7. The drill of claim 6 further comprising an insert rejection arm extending from at least one of the fingers such that upon spreading of the fingers the arm urges the insert out of the pocket.

8. The drill of claim 1 where the main body includes a radial passageway extending between the exterior of the main body and the insert pocket and generally aligned between the fingers.

9. The drill of claim 8 further comprising a cam wrench disposed in the radial passageway where the fingers are urged away from each other by rotation of the wrench.

10. The drill of claim 9 where the insert is urged out of the insert pocket by rotation of the wrench.

11. A modular drill comprising:
a main body having an attachment end for attachment to a toolholder and a receiving end defining an insert pocket and having one or more clamping fingers disposed in the pocket for holding an insert, the insert having a working end and a retention end, the retention end including a retention bit in the insert pocket retaining the retention bit in tension with the one or more clamping fingers, where at least one of the main body or one of the one or more clamping fingers includes a threaded aperture formed therein; and
a screw disposed in threaded engagement in the aperture, where turning of the screw in one direction results in contact of the screw with at least one of the one or more clamping fingers spreading the one or more fingers apart to release the retention bit of the insert retained in the insert pocket from the clamping finger.

12. The drill of claim 11 where the clamping fingers terminate in radially extending engagement surfaces and where at least one of the fingers includes an insert rejection arm extending from the one of the fingers.

13. The drill of claim 11 where the main body defines a longitudinal axis extending between the attachment end and the receiving end and includes at least two diametrically opposed circumferential outer main portions extending along the longitudinal axis with at least two diametrically opposed flutes disposed between the outer main portions where the fingers generally extend along the longitudinal axis within the main portions, and where the main body includes a radial passageway formed in one of the main portions and extends between the exterior of the main body and the insert pocket and is generally aligned with the threaded aperture.

14. The drill of claim 11 where the fingers are unitarily formed with the main portions of the main body.

15. The drill of claim 13 where fluid passageways are formed in the main portions of the body and generally extend along the longitudinal axis.

16. A modular drill comprising:
a main body having an attachment end for attachment to a toolholder and a receiving end defining an insert pocket and having one or more clamping fingers disposed in the pocket for holding an insert, the insert having a working end and a retention end, the retention end including a retention bit in the insert pocket retaining the retention bit in tension with the one or more clamping fingers, the main body including a radial passageway extending between the exterior of the main body and the insert pocket and generally aligned between the fingers for receiving a cam wrench for urging the fingers away from each other.

17. The drill of claim 16 further comprising a cam wrench disposed in the radial passageway where the fingers are urged away from each other by rotation of the wrench.

18. The drill of claim 17 where the cam wrench includes at least one off set end portion for engagement of at least one of the fingers during rotation.

19. The drill of claim 17 where the cam wrench includes at least one off set end portion for engagement of a retention bit of an insert disposed in the insert pocket during rotation.

20. The drill of claim 16 where the main body defines a longitudinal axis extending between the attachment end and the receiving end and includes at least two diametrically opposed circumferential outer main portions extending along the longitudinal axis with at least two diametrically opposed flutes disposed between the outer main portions where the fingers generally extend along the longitudinal axis within the main portions, and where the radial passageway formed in one of the flutes.
